(19) United States
(12) Patent Application Publication
(10) Pub. No.: US 2026/0334760 A1
(43) Pub. Date: Oct. 29, 2026

(54) DISTAL TIPS FOR MEDICAL DEVICES

(71) Applicant: Boston Scientific Medical Device Limited, Galway (IE)

(72) Inventor: Jianxin FANG, Suzhou (CN)

(73) Assignee: Boston Scientific Medical Device Limited, Galway (IE)

(21) Appl. No.: 19/659,076

(22) Filed: Apr. 27, 2026

(30) Foreign Application Priority Data
Apr. 29, 2025 (CN) 202510564871.4

(51) Int. Cl. A61B 1/00; A61B 1/018; A61B 1/05; A61B 1/06

(52) U.S. Cl. CPC A61B 1/00098; A61B 1/018; A61B 1/05; A61B 1/0676

(57) ABSTRACT

A distal tip of a medical device comprises a distal tip housing, an imaging device, a lighting element, and an elevator. A wire coupled to the elevator is configured to raise or lower the elevator. The distal tip housing includes a ledge. In a fully lowered configuration of the elevator, at least a portion of the wire rests on at least a portion of the ledge.

### CROSS-REFERENCE TO RELATED APPLICATIONS

This application claims the benefit of priority to Chinese Application No. 202510564871.4, filed on Apr. 29, 2025, which is incorporated by reference herein in its entirety.

### TECHNICAL FIELD

Various aspects of this disclosure relate generally to distal tips for medical devices. In particular, aspects of this disclosure pertain to distal tips for duodenoscopes, among other aspects.

### BACKGROUND

Duodenoscopes may include a handle and a sheath or shaft, and the shaft may be insertable into a body lumen of a subject. The sheath may terminate in a distal tip portion, which may include features, such as optical elements (e.g., camera, lighting, etc.), air/water outlets, and working channel openings. An elevator may be disposed at a distal tip and may be actuatable in order to change an orientation of a medical device/tool passed through the working channel. For example, the elevator may be pivotable or otherwise movable. During an endoscopic retrograde cholangiopancreatography (ERCP) procedure, an operator may insert a shaft of a duodenoscope into a body lumen and navigate the distal tip to a procedure site. Such body lumens may have small sizes, which may pose challenges to advancement of conventional devices.

### SUMMARY

Aspects of the disclosure relate to a distal tip of a medical device comprising a distal tip housing, an imaging device, a lighting element, and an elevator. A wire coupled to the elevator may be configured to raise or lower the elevator. The distal tip housing may include a ledge. In a fully lowered configuration of the elevator, at least a portion of the wire may rest on at least a portion of the ledge.

Any of the aspects disclosed herein may have any of the following features, alone or in any combination. The ledge may be slanted from a proximal end of the ledge to a distal end of the ledge. The ledge may be straight and slanted downward from the proximal end of the ledge to a distal end of the ledge. The distal tip housing may include an arm. The arm may include the ledge. The ledge may extend to a distalmost end of the arm. The arm may be a first arm. The distal tip housing may include a second arm housing the imaging device and the lighting element. The second arm may extend further distally than the first arm. The elevator may include a coupler defining an opening for receiving the wire. A distalmost surface of the coupler may be a distalmost surface of the elevator. The coupler may be further from a first lateral side of the elevator than a second lateral side of the elevator. The elevator may include a guide surface for contacting a medical instrument. The guide surface may include a longitudinally extending recess. The recess may be substantially aligned with a center of the coupler. The distal tip housing may include a first recess, a second recess, and a middle portion between the first recess and the second recess. The first recess and the second recess may be recessed relative to the middle portion and portions of the distal tip housing adjacent to the first recess and the second recess. At least one of the first recess or the second recess may have a substantially planar proximal surface. The middle portion may have a substantially planar distal surface. In a raised configuration of the elevator, the middle portion and a guide surface of the elevator may be configured to secure a medical device therebetween. The elevator may include a body at a proximal end of the elevator. The body may define an opening. The distal tip may further include a pin extending through the opening. The elevator may be configured to pivot about the pin. The distal tip housing may define a notch. The body may be received in the notch. The pin may extend through portions of the distal tip housing adjacent to the notch. The body may be further from a first lateral side of the elevator than a second lateral side of the elevator.

### BRIEF DESCRIPTION OF THE DRAWINGS

The accompanying drawings, which are incorporated in and constitute a part of this specification, illustrate aspects this disclosure and together with the description, explain the principles of the disclosure.

FIGS. 1A and 1B depict an exemplary medical device.

FIG. 2 is a perspective view of an exemplary distal tip for use with the medical device of FIGS. 1A and 1B.

FIGS. 3A-3C depict an elevator of the distal tip of FIG. 2.

FIGS. 4A and 4B are perspective views of the distal tip of FIG. 2 in alternative configurations.

FIG. 5 is a top view of the distal tip of FIG. 2.

FIG. 6 depicts a portion of the distal tip of FIG. 2.

FIG. 7 depicts another portion of the distal tip of FIG. 2.

### DETAILED DESCRIPTION

Distal tips of medical devices, such as duodenoscopes, may be difficult to navigate through small body lumens (e.g., body lumens of pediatric subjects or body lumens with strictures). Disclosed is a distal tip of an exemplary medical device for use in such small body lumens. The disclosed distal tip may have a smaller diameter/width than conventional distal tips, so as to facilitate navigation through smaller anatomies. The disclosed distal tip may include an elevator that may be raised or lowered in order to change an orientation of a medical instrument that extends through a working channel of the medical device. As compared with elevators in larger duodenoscopes, the disclosed elevator may have a smaller length. A control wire coupled to the elevator may raise or lower the elevator. The control wire may be coupled to the elevator by a coupler at a distal end of the elevator. The elevator may pivot about a pivot pin extending through a pivot at a proximal end of the elevator. The coupler may extend across only a portion of a width of the elevator. The elevator may be asymmetrical to accommodate a small size of the distal tip.

The elevator may be “locked” in a raised configuration. In other words, side portions of the elevator may be received in recesses of a housing of the distal tip when the elevator is in a fully raised configuration. This “locked” configuration may allow for the elevator to be retained in a raised position and may secure a medical instrument extending along the elevator in a deflected configuration of the medical instrument, by pinning the medical instrument against a distally-facing portion of the tip.

As mentioned above, the elevator may have a shorter length than conventional elevators. The elevator and the housing of the distal tip may be particularly adapted to transmit the forces necessary to raise or lower this shorter elevator. For example, a distal position of the coupler that couples the wire to the elevator facilitates transmission of force with a greater lever length. The housing of the distal tip may have a ledge that supports the actuation wire. This ledge may be particularly advantageous given that the coupler is at a distal end of the elevator, far from a distal opening of a lumen through which the actuation wire extends.

The distal tip also has other features and advantages that are discussed herein.

It may be understood that both the foregoing general description and the following detailed description are exemplary and explanatory only and are not restrictive of the invention, as claimed. As used herein, the terms “comprises,” “comprising,” “includes,” “including,” “has,” “having,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “distal” refers to a direction away from an operator, and the term “proximal” refers to a direction toward an operator. The term “exemplary” is used in the sense of “example,” rather than “ideal.” The term “approximately,” or like terms (e.g., “substantially”), includes values +/−10% of a stated value. The term “or” includes “and/or.”

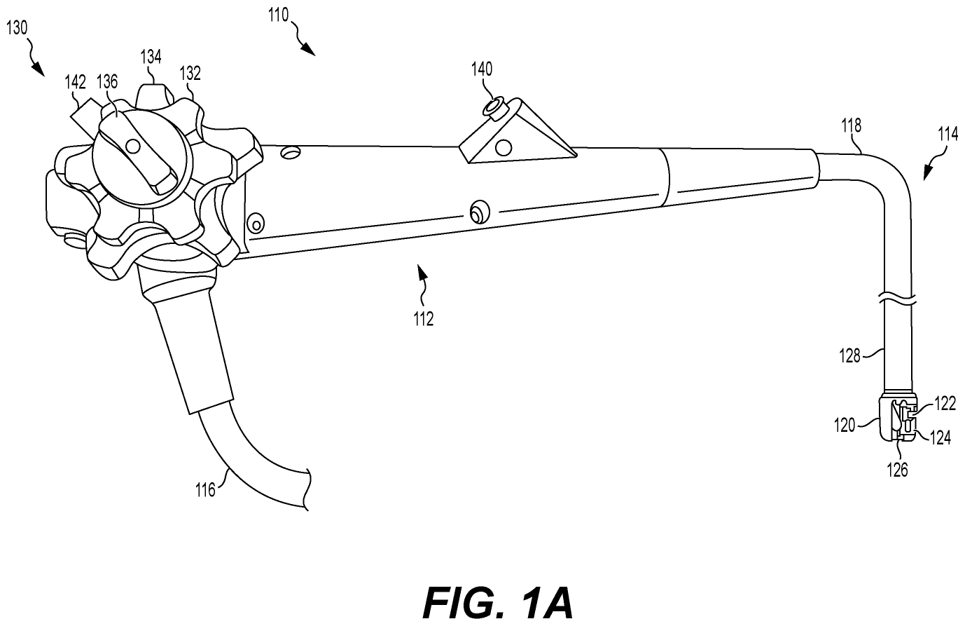

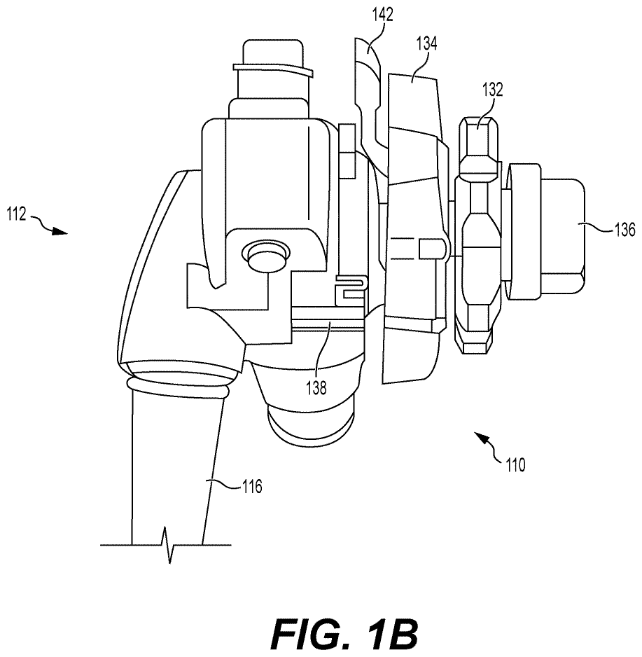

FIG. 1A depicts an exemplary medical device 110 having a handle 112 and an insertion portion 114. FIG. 1B shows a proximal end of handle 112. Medical device 110 may also include an umbilicus 116 for purposes of connecting medical device 110 to sources of, for example, air, water, suction, power, etc., as well as to image processing and/or viewing equipment. Although duodenoscopes and endoscopes (and combination devices that perform functions of duodenoscopes and endoscopes) are particularly referenced herein, the disclosure also encompasses other types of devices, such as bronchoscopes, gastroscopes, endoscopic ultrasound (“EUS”) scopes, colonoscopes, ureteroscopes, bronchoscopes, laparoscopes, cystoscopes, aspiration scopes, sheaths, catheters, or similar devices. A reference to a duodenoscope herein should be understood to encompass any of the above medical devices.

Insertion portion 114 may include a sheath or shaft 118 and a distal tip 120. Distal tip 120 may include an imaging device 122 (e.g., a camera) and a lighting element 124 (e.g., a light emitting diode (LED) or an optical fiber). Although the term “lighting element” is used herein, it will be appreciated that the term “lighting element” may include a plurality of lighting elements (e.g., a plurality of LEDs or optical fibers). Distal tip 120 may be side-facing. That is, imaging device 122 and lighting element 124 may face radially outward, perpendicularly, approximately perpendicularly, or otherwise transverse to a longitudinal axis of shaft 118 and distal tip 120 (e.g., at an angle of approximately 80 degrees to an angle of approximately 110 degrees). However, the disclosure is not limited to such an arrangement.

Distal tip 120 may also include an elevator 126 for changing an orientation of an accessory device or a tool inserted in a working channel of medical device 110. Elevator 126 may alternatively be referred to as a swing stand, pivot stand, raising base, or any suitable other term. Elevator 126 may be pivotable via, e.g., an actuation wire or another control element that extends from handle 112, through shaft 118, to elevator 126.

A distal portion of shaft 118 that is connected to distal tip 120 may have a steerable section 128. Steerable section 128 may be, for example, an articulation joint. Shaft 118 and steerable section 128 may include a variety of structures that are known or may become known in the art.

Handle 112 may have one or more actuators/control mechanisms 130. One or more of control mechanisms 130 may provide control over steerable section 128. One or more of control mechanisms 130 may allow for provision of air, water, suction, etc. For example, handle 112 may include control knobs 132, 134 for left, right, up, and/or down control of steerable section 128. For example, one of knobs 132, 134 may provide left/right control of steerable section 128, and the other of knobs 132, 134 may provide up/down control of steerable section 128. Handle 112 may further include one or more locking mechanisms 136 and 142 (e.g., knobs or levers) for preventing steering and/or braking of steerable section 128 in at least one of an up, down, left, or right direction. Handle 112 may include an elevator control lever 138 (see FIG. 1B). Elevator control lever 138 may raise and/or lower elevator 126, via connection between lever 138 and an actuating wire that extends from lever 138, through shaft 118, to elevator 126. A port 140 may allow passage of a tool through port 140, into a working channel of the medical device 110, through shaft 118, to distal tip 120.

In use, an operator may insert at least a portion of shaft 118 into a body lumen of a subject. Distal tip 120 may be navigated to a procedure site in the body lumen. For example, the operator may push or urge distal tip 120 distally in order to advance distal tip 120 through the body lumen. The operator may insert an accessory device (not shown) into port 140, and pass the accessory device through shaft 118 via a working channel to distal tip 120. The accessory device may exit the working channel at distal tip 120. The user may use elevator control lever 138 to raise elevator 126 and angle the accessory device toward a desired location (e.g., a papilla of the pancreatico-biliary tract). The user may use the accessory device to perform a medical procedure.

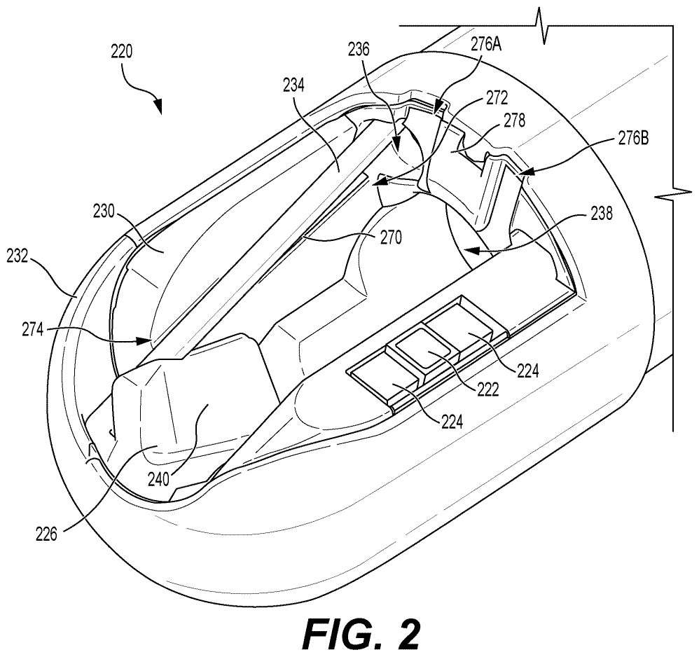

FIG. 2 shows a distal tip 220 that may be used in conjunction with medical device 110. For example, distal tip 220 may be used in place of distal tip 120. Distal tip 220 may have any of the features of distal tip 120, unless otherwise stated. Distal tip 220 may include a housing core 230 and a housing cover 232 partially covering housing core 230. Housing core 230 may house an imaging device 222 (having any property of imaging device 122) and lighting elements 224 (having any property of lighting element 124). Although two lighting elements 224 are shown, with one proximal of imaging device 222 and one distal of imaging device 222, such an arrangement is merely exemplary.

Distal tip 220 may also include an elevator 226. An actuation wire 234 may be coupled to elevator 226, thereby facilitating pivoting or rotating of elevator 226. Although the term “wire” is used, actuation wire 234 may be a cable, multi-stranded wire, or the like, and “wire” is meant to include any of such actuation elements. Actuation wire 234 may extend through a lumen of shaft 118 and may exit distally through an opening 236 of a lumen within distal tip 220 that communicated with the lumen of shaft 118. A medical instrument or device that is extended through a working channel of shaft 118 may exit distally via a working channel opening 238. The instrument or device may be extended along elevator 226. Actuation wire 234 may be actuated (e.g., using elevator control lever 138) in order to raise or lower elevator 226 in order to change an orientation of the medical instrument or device. Elevator 226 is described in further detail below.

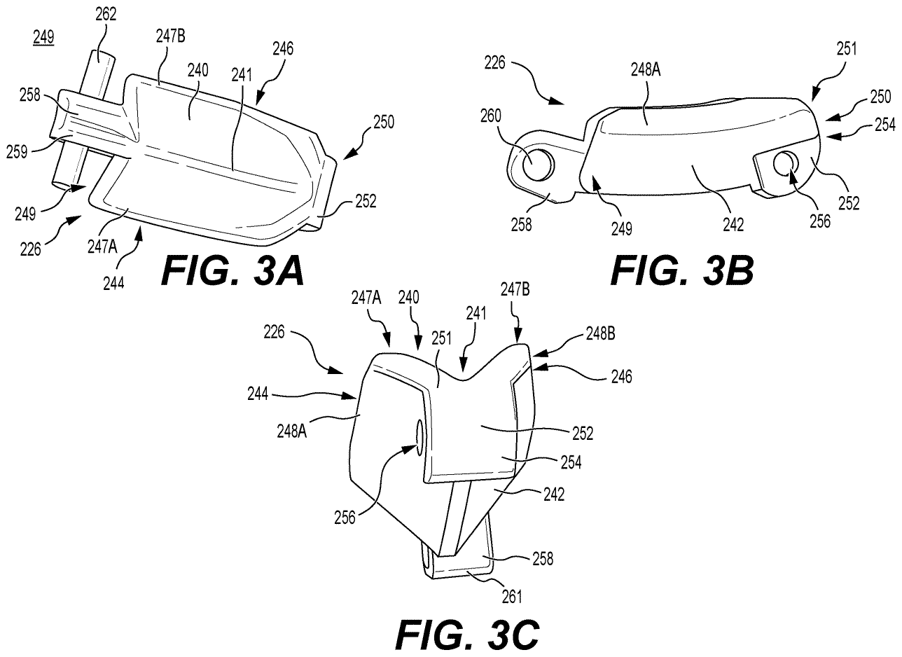

FIGS. 3A-3C depict elevator 226 in isolation from a remainder of distal tip 220 and show details of elevator 226. Elevator 226 may have a guide surface 240 for contacting a medical instrument or device and changing an orientation of the medical instrument or device. A medical instrument or device that is extended through working channel opening 238 may move along guide surface 240 and may contact guide surface 240. Guide surface 240 may include a recess 241 that extends longitudinally along a length of elevator 226. A medical device or instrument may be guided to recess 241 and, as discussed below, may be held in place in recess 241 when elevator 226 is in a raised/closed configuration. Guide surface 240 may extend from a proximal end 249 of elevator 226 to a distal end 250 of elevator 226.

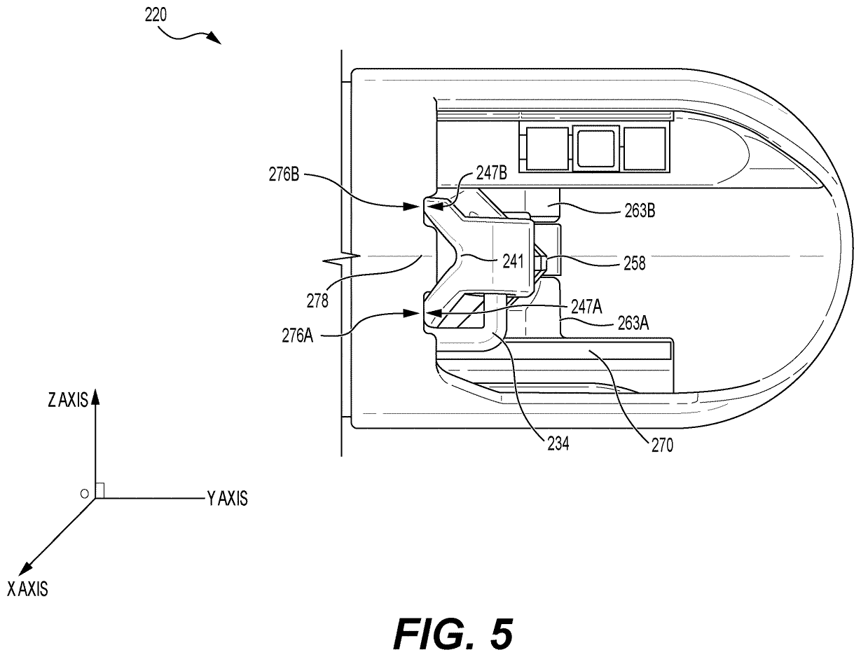

An opposite side of elevator 226 from guide surface 240 may have a back surface 242. Back surface 242 may face housing cover 232 and may not contact a medical device or instrument. Elevator 226 may include a first top surface 247a on a first lateral side 244 of elevator 226. Elevator 226 may include a second top surface 247b on a second lateral side 246. Second lateral side 246 may be a side that is closer to imaging device 222 and lighting elements 224. First lateral side 244 may be a side that is closer to actuation wire 234. The lateral direction is a “Z” direction in the coordinate system of FIG. 5. Top surface 247a and second top surface 247b may be adjacent to guide surface 240. Elevator 226 may have a first side surface 248a on first lateral side 244 and a second side surface 248b on second lateral side 246. First top surface 247a/second top surface 247b may extend between guide surface 240 and first side surface 248a/second side surface 248b, respectively. First side surface 248a/second side surface 248b may extend between back surface 242 and first top surface 247a/second top surface 247b, respectively. Elevator 226 may have a distal surface 251 at distal end 250.

Elevator 226 includes a coupler 252 at to distal end 250 of elevator 226. Coupler 252 may have a distal surface 254 that may be continuous with distal surface 251. In other words, distal surface 254 may form a part of a distalmost surface of elevator 226. Coupler 252 may be disposed on back surface 242 and may extend to a distalmost end of back surface 242. Coupler 252 may define an opening 256. Opening 256 may extend substantially perpendicularly to a longitudinal axis of elevator 226 (e.g., perpendicularly to recess 241). Opening 256 may extend laterally, substantially parallel to a line drawn from first lateral side 244 to second lateral side 246. Actuation wire 234 may extend into opening 256 and be fixedly coupled to coupler 252. For example, as shown in FIG. 2, actuation wire 234 may include an approximately 90-degree bend, such that a distalmost portion of actuation wire 234 extends into opening 256.

Elevator 226 includes a pivot body 258 at proximal end 249 of elevator 226. A top surface 259 (a surface along a same side of elevator 226 as guide surface 240) of pivot body 258 may be continuous with guide surface 240, so as to provide a smooth surface for receiving and guiding a medical device or instrument that extends out of working channel opening 238. In aspects, recess 241 may extend along top surface 259 of pivot body 258. A back surface 261 of pivot body 258 may extend further away from guide surface 240 than back surface 242 of other portions of elevator 226. In other words, as shown in FIGS. 3B and 3C, back surface 261 extends down from back surface 242. An opening 260 may extend substantially perpendicularly to a longitudinal axis of elevator 226 (e.g., perpendicularly to recess 241). Opening 260 may extend laterally, substantially parallel to a line drawn from first lateral side 244 to second lateral side 246. A pin 262 or other axle (shown in FIG. 3A) may extend through opening 260, and elevator 226 may be pivotable or rotatable about pin 262 extending through opening 260.

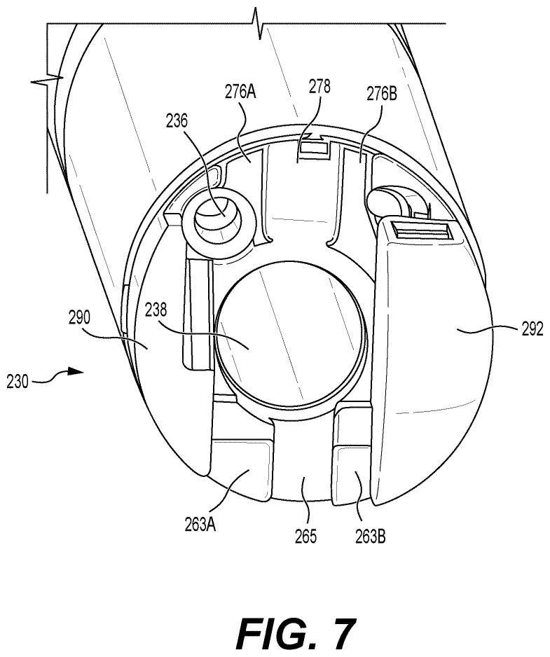

As shown particularly in FIGS. 5 and 7, pivot body 258 may be positioned in a notch 265 (FIG. 7) between protrusions 263a, 263b of housing core 230. Protrusions 263a, 263b may be adjacent to notch 265. Pin 262 may extend through protrusions 263a, 263b and through pivot body 258. Thus, as actuation wire 234 is moved proximally or distally, elevator 226 may pivot about pin 262.

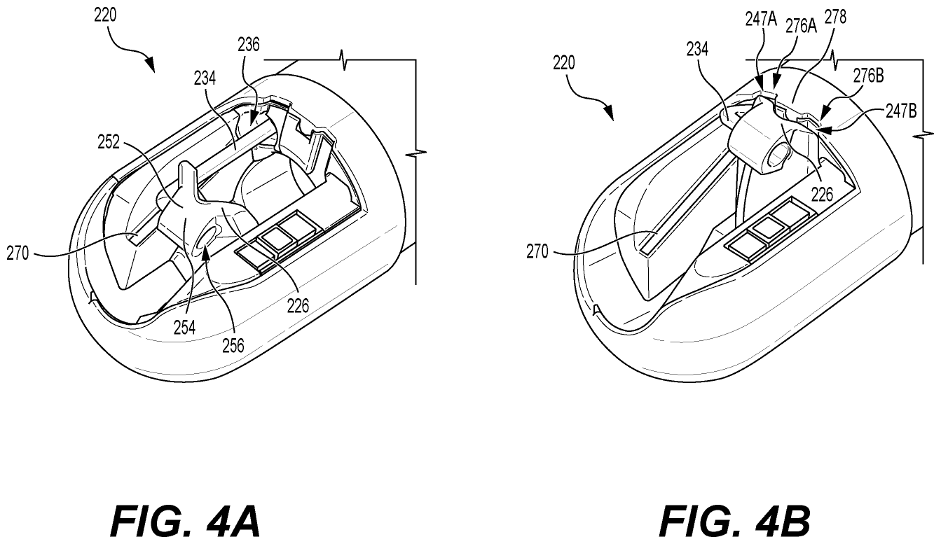

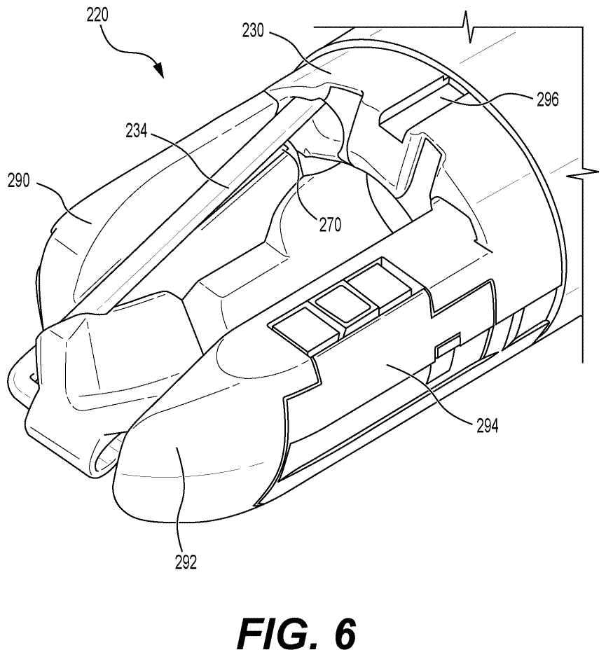

FIGS. 4A and 4B show perspective views of distal tip 220 with elevator 226 in a partially raised configuration (FIG. 4A) and a fully raised (closed) configuration (FIG. 4B). FIG. 5 shows a top view of distal tip 220 with elevator 226 fully raised. FIG. 6 shows distal tip 220 with housing cover 232 removed. FIG. 7 shows distal tip 220 with housing cover 232 removed, along with elevator 226 and actuation wire 234 removed, to reveal aspects of housing core 230. As shown particularly in FIGS. 6 and 7, housing core 230 may include a first arm 290 and a second arm 292. Imaging device 222 and lighting elements 224 may be housed within second arm 292. For example, imaging device 222 and lighting elements 224 may be disposed in a module 294, which may be fitted into second arm 292.

As shown particularly in FIGS. 2, 4A, 4B, and 6, a ledge 270 may extend longitudinally along first arm 290 of housing core 230. For example, ledge 270 may extend along an inner surface of first arm 290 (a surface facing second arm 292), to a distal end of first arm 290. Ledge 270 may have a proximal end 272 and a distal end 274, and may be a straight surface between those ends. Ledge 270 may slant downward from proximal end 272 toward distal end 274, where an upward direction is a direction defined by the general direction in which imaging device 222 and lighting elements 224 face. The up/down direction is the “X” axis (into/out of the page) in FIG. 5. In this way, ledge 270 is angled relative to the longitudinal axis of tip 220. Ledge 270 may support actuation wire 234. Support from ledge 270 may facilitate a relatively large distance between pivot body 258 and coupler 252. For example, ledge 270 may maintain an alignment of actuation wire 234 and may limit slack in actuation wire 234. Ledge 270 may slant downward due to opening 236 through which actuation wire 234 exits being higher than opening 256 of coupler 252 when elevator 226 is fully lowered (FIG. 2). In a fully lowered configuration of elevator 226 (FIG. 2), at least a portion of actuation wire 234 may rest on at least a portion of ledge 270. As elevator 226 is raised (FIGS. 4A and 4B), actuation wire 234 may lift away from ledge 270.

As shown in FIG. 6, second arm 292 may extend further distally than first arm 290. First arm 290 may have a relatively shorter longitudinal length than second arm 292 so as to allow for ledge 270 to extend an entire length of first arm 290 and off of a distalmost end of first arm 290, without interfering with movement of actuation wire 234 or elevator 226. For example, a portion of a length of actuation wire 234 may be distal of a distalmost end of ledge 270 (and first arm 290). First arm 290 may be sufficiently longitudinally long so as to provide support to actuation wire 234 without inhibiting movement of actuation wire 234.

As shown particularly in FIGS. 3A-3C and 5, elevator 226 may be asymmetrical about a central longitudinal axis of elevator 226. For example, recess 241 of guide surface 240 may be closer to second lateral side 246 than to first lateral side 244. Recess 241 may extend substantially parallel to the central longitudinal axis of elevator 226. In aspects, first top surface 247a and second top surface 247b may be approximately a same height relative to recess 241. Thus, a side of guide surface 240 that is closer to second lateral side 246 may be steeper than a side of guide surface 240 that is closer to first lateral side 244. Coupler 252 may be closer to second lateral side 246 than to first lateral side 244. In other words, coupler 252 may be further from first lateral side 244 than second lateral side 246. In aspects, recess 241 may be approximately laterally centered with respect to coupler 252 (a center of coupler 252 may be approximately aligned with recess 241). Pivot body 258 may also be closer to second lateral side 246 than to first lateral side 244. In other words, pivot body 258 may be further from first lateral side 244 than from second lateral side 246. In some aspects, as shown particularly in FIG. 3A, a longitudinal centerline of pivot body 258 may be closer to second lateral side 246 than a longitudinal centerline of coupler 252. Thus, recess 241 may be off-centered with respect to pivot body 258.

The arrangements above may be particularly adapted for the small size of distal tip 220. For example, the asymmetrical arrangement may accommodate a position and size of opening 236 through which actuation wire 234 passes and a position of a ledge 270 for supporting actuation wire 234. In aspects, a width of guide surface 240 from first lateral side 244 to second lateral side 246 may be approximately the same as a width of working channel opening 238 and may be approximately aligned with working channel opening 238. As shown particularly in FIG. 7, opening 236 for actuation wire 234 may partially laterally overlap working channel opening 238. In other words, a line drawn from the top to the bottom of distal tip 220 (along the X axis shown in FIG. 5) may extend through both opening 236 and working channel opening 238. This arrangement may allow the required components and lumen sizes to be accommodated in a smaller distal tip 220. For example, opening 236 may be positioned at least partially above (in the X-direction shown in FIG. 5) working channel opening 238 so as to accommodate both working channel opening 238 and opening 236 in small distal tip 220. For example, working channel opening 238 may need to be a same or similar size to a working channel of a conventional duodenoscope to accommodate medical instruments or devices. Thus, notch 265 between protrusions 263a, 263b may be off-centered with respect to working channel opening 238 (on a side of a centerline of working channel opening 238 that is closer to imaging device 222 and lighting elements 224) so as to accommodate actuation wire 234 as it runs down ledge 270, along with the bend in actuation wire 234 as it enters opening 256 of coupler 252.

An arrangement of actuation wire 234, ledge 270, pivot body 258 (and pin 262), and coupler 252 (including opening 256) may be particularly adapted to provide the necessary torque to actuate elevator 226 without requiring excess force on elevator control lever 138 by an operator. For example, the arrangement of the elements above may allow for actuation of elevator 226, which may have a smaller length than conventional elevators, without undue force by an operator on elevator control lever 138.

Housing core 230 and housing cover 232 may also include features to help to secure an instrument or medical device inserted through opening 238 when elevator 226 is in a raised configuration. Housing core 230 and housing cover 232 may together define a first recess 276a and a second recess 276b. First recess 276a and second recess 276b may each be recessed proximally relative to surrounding (e.g., adjacent) portions of housing core 230 and housing cover 232. A middle portion 278 may be between first recess 276a and second recess 276b. First recess 276a and second recess 276b may be recessed relative to middle portion 278. Middle portion 278 may be approximately longitudinally aligned with portions of housing core 230 and housing cover 232 that are surrounding first recess 276a and second recess 276b. In other words, middle portion 278 may not extend or protrude further distally than portions of housing core 230 and housing cover 232 surrounding first recess 276a and second recess 276b. Housing core 230 may include a groove 296 along middle portion 278 to mate with a complementary protrusion on housing cover 232.

First recess 276a and second recess 276b may have an approximately rectangular cross-sectional shape. For example, proximal surfaces defining first recess 276a and second recess 276b may be approximately straight or flat (e.g., planar) and may extend substantially laterally (in the Z-direction of the coordinate axis of FIG. 5). Middle portion 278 may also have a substantially straight or flat (e.g., planar) distal surface.

As shown in FIGS. 4B and 5, when elevator 226 is fully raised (closed), first top surface 247a and second top surface 247b may be received in first recess 276a and second recess 276b, respectively. Shapes of first top surface 247a and second top surface 247b may be complementary with shapes of first recess 276a and second recess 276b, respectively. A medical device or medical instrument extended through opening 238 may be retained between recess 241 of guide surface 240 and middle portion 278. A distance between recess 241 and middle portion 278, when elevator 226 is fully raised) may be chosen to accommodate common diameters of medical instruments or devices. In other words, middle portion 278 and recess 241 may be configured to secure the medical device or instrument therebetween. Pinning a medical device or medical instrument between recess 241 (or another portion of guide surface 240) and middle portion 278 may be referred to as “locking” elevator 226. The arrangement of first recess 276a, second recess 276b, and middle portion 278 may be particularly adapted to the smaller sized distal tip 220 and elevator 226 disclosed herein.

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Additionally, a variety of elements from each of the presented examples can be combined to achieve a same or similar result as one or more of the disclosed embodiments. Thus, the features of the examples should not be viewed as being mutually exclusive or applicable to any particular distal tip and may be combined in any suitable manner. Accordingly, the invention is not to be considered as limited by the foregoing description.

1. A distal tip of a medical device comprising:
a distal tip housing;
an imaging device;
a lighting element; and
an elevator, wherein a wire coupled to the elevator is configured to raise or lower the elevator;
wherein the distal tip housing includes a ledge, wherein, in a fully lowered configuration of the elevator, at least a portion of the wire rests on at least a portion of the ledge.

2. The distal tip of claim 1, wherein the ledge is slanted from a proximal end of the ledge to a distal end of the ledge.

3. The distal tip of claim 2, wherein the ledge is straight and slanted downward from the proximal end of the ledge to a distal end of the ledge.

4. The distal tip of claim 1, wherein the distal tip housing includes an arm, wherein the arm includes the ledge, and the ledge extends to a distalmost end of the arm.

5. The distal tip of claim 4, wherein the arm is a first arm, wherein the distal tip housing includes a second arm housing the imaging device and the lighting element, wherein the second arm extends further distally than the first arm.

6. The distal tip of claim 1, wherein the elevator includes a coupler defining an opening for receiving the wire, wherein a distalmost surface of the coupler is a distalmost surface of the elevator.

7. The distal tip of claim 6, wherein the coupler is further from a first lateral side of the elevator than a second lateral side of the elevator.

8. The distal tip of claim 7, wherein the elevator includes a guide surface for contacting a medical instrument, wherein the guide surface includes a longitudinally extending recess, and wherein the recess is substantially aligned with a center of the coupler.

9. The distal tip of claim 1, wherein the distal tip housing includes a first recess, a second recess, and a middle portion between the first recess and the second recess, wherein the first recess and the second recess are recessed relative to the middle portion and portions of the distal tip housing adjacent to the first recess and the second recess.

10. The distal tip of claim 9, wherein at least one of the first recess or the second recess has a substantially planar proximal surface.

11. The distal tip of claim 9, wherein the middle portion has a substantially planar distal surface.

12. The distal tip of claim 9, wherein, in a raised configuration of the elevator, the middle portion and a guide surface of the elevator are configured to secure a medical device therebetween.

13. The distal tip of claim 1, wherein the elevator includes a body at a proximal end of the elevator, wherein the body defines an opening, and wherein the distal tip further includes:
a pin extending through the opening, wherein the elevator is configured to pivot about the pin.

14. The distal tip of claim 13, wherein the distal tip housing defines a notch, wherein the body is received in the notch, and wherein the pin extends through portions of the distal tip housing adjacent to the notch.

15. The distal tip of claim 13, wherein the body is further from a first lateral side of the elevator than a second lateral side of the elevator.

16. A distal tip of a medical device comprising:
a distal tip housing;
an imaging device;
a lighting element; and
an elevator, wherein the elevator includes a coupler defining an opening, wherein a distalmost surface of the coupler is a distalmost surface of the elevator, wherein a wire is received within the opening to couple the wire to the elevator, and wherein the wire is configured to raise or lower the elevator.

17. The distal tip of claim 16, wherein the coupler is further from a first lateral side of the elevator than a second lateral side of the elevator.

18. The distal tip of claim 17, wherein the elevator includes a guide surface for contacting a medical instrument, wherein the guide surface includes a longitudinally extending recess, and wherein the recess is substantially aligned with a center of the coupler.

19. A distal tip of a medical device comprising:
a distal tip housing, wherein the distal tip housing includes a first recess, a second recess, and a middle portion between the first recess and the second recess, wherein the first recess and the second recess are recessed relative to the middle portion and portions of the distal tip housing adjacent to the first recess and the second recess;
an imaging device;
a lighting element; and
an elevator, wherein a wire coupled to the elevator is configured to raise or lower the elevator, wherein, in a raised configuration of the elevator, the middle portion and a guide surface of the elevator are configured to secure a medical device therebetween.

20. The distal tip of claim 19, wherein at least one of the first recess or the second recess has a substantially planar proximal surface, and wherein the middle portion has a substantially planar distal surface.
